(19) United States
(12) Patent Application Publication
(10) Pub. No.: US 2023/0372161 A1
(43) Pub. Date: Nov. 23, 2023

(54) ABSORBENT ARTICLE, ABSORBENT SYSTEM AND DISPENSING SYSTEM

(71) Applicant: 3M INNOVATIVE PROPERTIES COMPANY, St. Paul, MN (US)

(72) Inventors: Bradley D. Craig, Lake Elmo, MN (US); Saurabh Batra, Minneapolis, MN (US); Paul J. Homnick, Lake Elmo, MN (US); Jamie Vadnais, Cottage Grove, MN (US); Satinder K. Nayar, Woodbury, MN (US)

(21) Appl. No.: 18/031,228

(22) PCT Filed: Oct. 15, 2021
(86) PCT No.: PCT/IB2021/059518

§ 371 Date: Apr. 11, 2023

Related U.S. Application Data
(60) Provisional application No. 63/198,760, filed on Nov. 11, 2020.

(51) Int. Cl. A61F 13/36; A61F 13/20; A61F 13/15; A61F 15/00; A61C 17/08

(52) U.S. Cl. CPC A61F 13/36; A61F 13/2008; A61F 13/15203; A61F 15/001; A61C 17/08

(57) ABSTRACT

An absorbent article includes at least two absorbent pads spaced apart from each other. The absorbent article further includes an interconnect member. Each absorbent pad includes an absorbent material configured to absorb a liquid. Each absorbent pad further includes an outer surface. The interconnect member joins the outer surfaces of the at least two absorbent pads. The interconnect member is stretchable.

### TECHNICAL FIELD

The present disclosure refers to an absorbent article, an absorbent system including a plurality of absorbent articles, and a dispensing system including the absorbent system.

### BACKGROUND

Isolation of a working area and management of a dental fluid, such as moisture, blood, and saliva, in a mouth of a patient is generally a challenge for dental practitioners. Multiple approaches have been taken over the years to address this challenge. Conventional solutions, for example, rubber dams, or cotton rolls, have various drawbacks, such as discomfort for the patient, difficulty of placement in the mouth of the patient, or eventual saturation, and a need to be frequently replaced. Generally, the rubber dams are commonly used by the dental practitioners. However, the rubber dams are difficult to use and can cause discomfort to the patient. Therefore, the rubber dams are utilized in only about 15% of clinical cases. Further, the cotton rolls are typically positioned between a tooth and cheek, or between the teeth and a tongue of the patient. The cotton rolls get saturated rapidly. Further, the cotton rolls may become slippery and uncomfortable to work with once saturated. Therefore, the cotton rolls may need to be frequently replaced.

Thus, it may be desirable to have a simple, comfortable, and easy to set up solution to isolate the working area and manage the dental fluid in the mouth of the patient.

### SUMMARY

Generally, the present disclosure relates to an absorbent article for an oral environment. The present disclosure also relates to an absorbent system including a plurality of absorbent articles, and a dispensing system including a dispenser.

A first aspect of the present disclosure provides an absorbent article. The absorbent article includes at least two absorbent pads spaced apart from each other. Each absorbent pad includes an absorbent material configured to absorb a liquid. Each absorbent pad further includes an outer surface. The absorbent article further includes an interconnect member. The interconnect member joins the outer surfaces of the at least two absorbent pads. The interconnect member is stretchable.

A second aspect of the present disclosure provides an absorbent system. The absorbent system includes a plurality of absorbent articles. Each of the plurality of absorbent articles includes at least two absorbent pads spaced apart from each other. Each absorbent pad includes an absorbent material configured to absorb a liquid. Each absorbent pad further includes an outer surface. Each of the plurality of absorbent articles further includes an interconnect member joining the outer surfaces of the at least two absorbent pads. The interconnect member is stretchable. The absorbent system further includes at least one line of weakness. The at least one line of weakness joins two adjacent absorbent articles of the plurality of absorbent articles.

A third aspect of the present disclosure provides a dispensing system. The dispensing system includes an absorbent system and a dispenser. The absorbent system includes a plurality of absorbent articles. Each of the plurality of absorbent articles includes at least two absorbent pads spaced apart from each other. Each absorbent pad includes an absorbent material configured to absorb a liquid. Each absorbent pad further includes an outer surface. Each of the plurality of absorbent articles further includes an interconnect member joining the outer surfaces of the at least two absorbent pads. The interconnect member is stretchable. The absorbent system further includes at least one line of weakness. The at least one line of weakness joins two adjacent absorbent articles of the plurality of absorbent articles. The dispenser at least partially receives the absorbent system therein, such that at least one absorbent article of the plurality of absorbent articles at least partially extends outwardly from the dispenser.

### BRIEF DESCRIPTION OF DRAWINGS

Exemplary embodiments disclosed herein may be more completely understood in consideration of the following detailed description in connection with the following figures. The figures are not necessarily drawn to scale. Like numerals used in the figures refer to like components. When pluralities of similar elements are present, a single reference numeral may be assigned to each plurality of similar elements with a small letter designation referring to specific elements. When referring to the elements collectively or to a non-specific one or more of the elements, the small letter designation may be eliminated. However, it will be understood that the use of a numeral to refer to a component in a given figure is not intended to limit the component in another figure labeled with the same number.

FIG. 1A illustrates a schematic top view of an absorbent article, according to an embodiment of the present disclosure;

FIG. 1B illustrates a schematic perspective view of the absorbent article, according to an embodiment of the present disclosure;

FIG. 2A illustrates a schematic cross-sectional view of an absorbent pad, according to an embodiment of the present disclosure;

FIG. 2B illustrates a schematic top view of the absorbent pad of FIG. 2A, according to an embodiment of the present disclosure;

FIG. 3 illustrates a schematic cross-sectional view of another absorbent article, according to an embodiment of the present disclosure;

FIG. 4A illustrates a schematic top view of another absorbent article, according to an embodiment of the present disclosure;

FIG. 4B illustrates a schematic perspective view of the absorbent article of FIG. 4A, according to an embodiment of the present disclosure;

FIG. 5A illustrates a schematic top view of another absorbent article, according to an embodiment of the present disclosure;

FIG. 5B illustrates a schematic perspective view of the absorbent article of FIG. 5A, according to an embodiment of the present disclosure;

FIG. 6A illustrates a schematic top view of a lower jaw with an absorption article, according to an embodiment of the present disclosure;

FIG. 6B illustrates a schematic top view of the lower jaw with another absorption article, according to an embodiment of the present disclosure;

FIGS. 7A-7G illustrate schematic views of various absorption pads, according to some embodiments of the present disclosure;

FIGS. 8A-8C illustrate schematic views of another absorbent article, according to an embodiment of the present disclosure;

FIGS. 9A-9C illustrate schematic views of various absorption pads, according to some embodiments of the present disclosure;

FIG. 10 illustrates a schematic view of another absorption article, according to an embodiment of the present disclosure;

FIG. 11A illustrates a schematic perspective view of another absorption article, according to an embodiment of the present disclosure;

FIG. 11B illustrates a schematic sectional view of the absorption article taken along a line 1-1 of FIG. 11A;

FIG. 11C illustrates a schematic front view of the absorption article of FIG. 11A;

FIG. 11D illustrates a schematic rear view of the absorption article of FIG. 11A;

FIG. 11E illustrates a schematic top view of the absorption article of FIG. 11A;

FIG. 12 illustrates a schematic sectional side view of another absorption article, according to an embodiment of the present disclosure;

FIG. 13A illustrates a schematic sectional side view of another absorption article, according to an embodiment of the present disclosure;

FIG. 13B illustrates a schematic top view the absorption article of FIG. 13A;

FIG. 14A illustrates a schematic sectional side view of another absorption article, according to an embodiment of the present disclosure;

FIG. 14B illustrates a schematic sectional side view of another absorption article, according to an embodiment of the present disclosure;

FIGS. 15A and 15B illustrate schematic top views of different absorbent systems, according to some embodiments of the present disclosure; and

FIG. 16 illustrates a schematic perspective view of a dispensing system, according to an embodiment of the present disclosure.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

As recited herein, all numbers should be considered modified by the term “about”. As used herein, “a,” “an,” “the,” “at least one,” and “one or more” are used interchangeably. As used herein, layers, components, or elements may be described as being adjacent to one another. Layers, components, or elements can be adjacent one another by being in direct contact, by being connected through one or more other components, or by being held next to one another or attached to one another. Layers, components, or elements that are in direct contact may be described as being immediately adjacent.

The present disclosure relates to an absorbent article including at least two absorbent pads spaced apart from each other and an interconnect member. Each absorbent pad includes an absorbent material configured to absorb a liquid. Each absorbent pad further includes an outer surface. The interconnect member joins the outer surfaces of the at least two absorbent pads. The interconnect member is stretchable. The present disclosure further relates to an absorbent system including a plurality of absorbent articles and at least one line of weakness. The present disclosure further relates to a dispensing system including the absorbent system and a dispenser.

The absorbent article of the present disclosure may be used to isolate a working area and to absorb the liquid in an oral environment within a mouth of a patient. The liquid may include saliva, water, and blood. The absorbent article may further prevent the liquid from entering a throat of the patient. Conventional methods of isolation of the working area or absorption of the liquid in the oral environment include use of rubber dams, cotton rolls, or suction devices. Conventional methods have various drawbacks, such as discomfort for the patient, difficulty of placement in the mouth of the patient, or eventual saturation, and a need to be frequently replaced.

The absorbent pads may be molded into a desired shape to fit in the mouth of the patient. The absorbent pads may further be formable. Therefore, the absorbent pads may fit in the mouth of the patient without causing any discomfort while providing an efficient absorption of the liquid from the oral environment. Further the absorbent pads may have curved or rounded edges. The curved edges may prevent any bruises or injures to oral tissues in the oral environment of the patient.

When using the absorbent articles, a dental clinician may penetrate or puncture the interconnect member to form a through hole or a slit and place the interconnect member over a tooth in the working area. As the interconnect member is stretchable, the through hole or the slit may surround and secure the tooth. The interconnect member of the absorbent article may therefore help to isolate the working area and may provide the dental clinician with visibility and accessibility to the working area.

The absorbent article of the present disclosure may be simple to manufacture, comfortable to the patient, and easy to set up. The absorbent article of the present disclosure can be used to isolate the working area and to absorb the liquid in the oral environment.

Further, the dental clinician may detach or tear one or more absorbent articles from the plurality of absorbent articles. For ease of detaching or tearing, the absorbent system may have at least one line of weakness.

In addition, the dispensing system may further provide ease and convenience to the dental clinician to dispense the absorbent articles, according to application requirements.

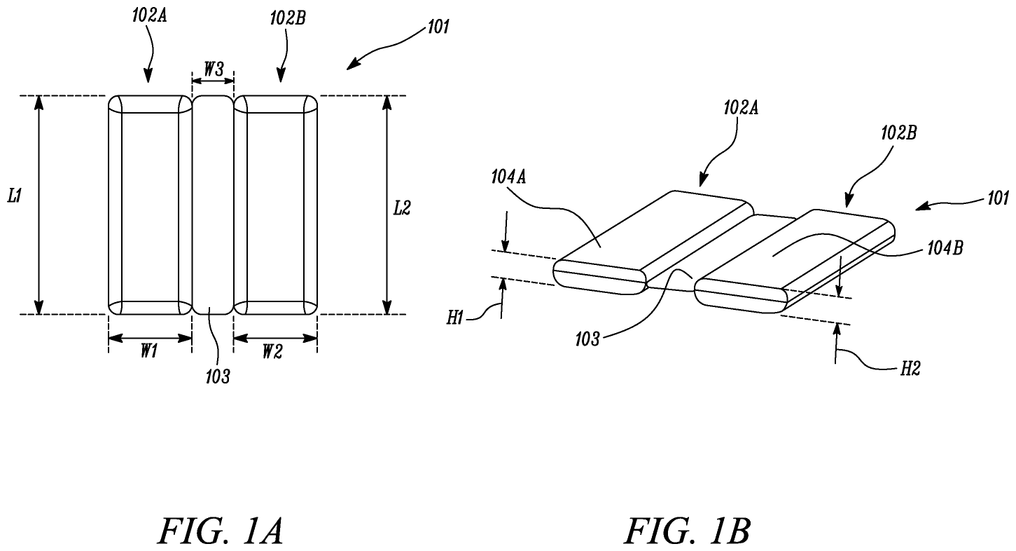

FIGS. 1A and 1B illustrate a schematic top view and a schematic perspective view, respectively, of an absorbent article 101. The absorbent article 101 may be used by a dental clinician to keep an oral environment dry. The dental clinician may be a professional specialized in addressing an oral health of a patient. The absorbent article 101 includes at least two absorbent pads 102A, 102B spaced apart from each other. Each absorbent pad 102A, 102B includes an outer surface 104A, 104B. Specifically, the absorbent pad 102A include the outer surface 104A. The absorbent pad 102B includes the outer surface 104B.

Each absorbent pad 102A, 102B further includes an absorbent material configured to absorb a liquid. In some embodiments, the liquid includes at least one of saliva, water, and blood. In some embodiments, the absorbent material is made of one or more superabsorbent particles, superabsorbent fibers, cellulose ethyl sulfonate, carboxymethyl cellulose, sodium polyacrylate, hydrogel particles, hydrogel-based non-woven material, cellulose foam, hydrogel polyurethane, silicone foam and polyurethane foam. As used herein, the term “absorbent material” refers to a material with an ability to absorb a liquid (such as, saliva, water, and blood). The absorbent material may have a suitable absorption rate as per desired application attributes. As used herein, the term “absorption rate” refers to a volume of liquid absorbed per unit time, for example, milliliters per second. In some embodiments, the absorption of the liquid may not significantly increase a size or a volume of the absorbent pads 102A, 102B. However, in some other embodiments, the absorption of the liquid may increase the size or the volume of the absorbent pads 102A, 102B.

The absorbent article 101 further includes an interconnect member 103. The interconnect member 103 joins the outer surfaces 104A, 104B of the at least two absorbent pads 102A, 102B. In some embodiments, the interconnect member 103 joins the outer surfaces 104A, 104B of the at least two absorbent pads 102A, 102B by one or more of an adhesive, heat sealing, stitching, or frictional heating.

Furthermore, the interconnect member 103 is stretchable. In some embodiments, the interconnect member 103 is made of an elastomeric material. In some embodiments, the elastomeric material is rubber. In some embodiments, the interconnect member 103 is made of a liquid impermeable material. In some embodiments, the interconnect member 103 includes at least one of polyurethane, styrene block copolymers, polyester elastomers, and elastomeric polyolefins. In some embodiments, the interconnect member 103 is made of a non-woven material.

In some embodiments, each absorbent pad 102A, 102B has a length L1, L2. Specifically, the absorbent pad 102A has the length L1. The absorbent pad 102B has the length L2. In some embodiments, the lengths L1, L2 of the absorbent pads 102A, 102B may be less than about 1 centimeter (cm), less than about 2 cm, less than about 3 cm, less than about 4 cm, less than about 5 cm, less than about 5.5 cm, or as per desired application attributes. In the illustrated embodiment of FIG. 1A, the lengths L1, L2 of the absorbent pads 102A, 102B are substantially equal to each other. However, in some other embodiments, the lengths L1, L2 of the absorbent pads 102A, 102B may be different from each other. The lengths L1, L2 of absorbent pads 102A, 102B may be such that each absorbent pad 102A, 102B is easy to fit in the oral environment as well as comfortable for the patient.

In some embodiments, each absorbent pad 102A, 102B has a width W1, W2. Specifically, the absorbent pad 102A has the width W1. The absorbent pad 102B has the width W2. In some embodiments, the widths W1, W2 of the absorbent pads 102A, 102B may be greater than about 0.5 cm, greater than about 1 cm, greater than about 2 cm, greater than about 3 cm, or as per desired application attributes. In the illustrated embodiment of FIG. 1A, the widths W1, W2 of the absorbent pads 102A, 102B are substantially equal to each other. However, in some other embodiments, the widths W1, W2 of the absorbent pads 102A, 102B may be different from each other. The widths W1, W2 of absorbent pads 102A, 102B may be such that each absorbent pad 102A, 102B is easy to fit in the oral environment as well as comfortable for the patient.

In some embodiments, each absorbent pad 102A, 102B has a height H1, H2. Specifically, the absorbent pad 102A has the height H1. The absorbent pad 102B has the height H2. In some embodiments, the heights H1, H2 of the absorbent pads 102A, 102B may be greater than about 2 millimeters (mm), greater than about 4 mm, greater than about 5 mm, greater than about 6 mm, greater than about 8 mm, greater than about 10 mm, greater than about 12 mm, or as per desired application attributes. In the illustrated embodiment of FIG. 1B, the heights H1, H2 of the absorbent pads 102A, 102B are substantially equal to each other. However, in some other embodiments, the heights H1, H2 of the absorbent pads 102A, 102B may be different. The height H1, H2 of each absorbent pad 102A, 102B may be such that each absorbent pad 102A, 102B is easy to fit in the oral environment as well as comfortable for the patient.

In the illustrated embodiment of FIG. 1A, each of the absorbent pads 102A, 102B has a cuboidal shape. However, the absorbent pads 102A, 102B may have any suitable shape as per desired application attributes. In some embodiments, a shape of each absorbent pad 102A, 102B is at least one of cylindrical, pyramidal, cuboidal, and polyhedral.

In some embodiments, the interconnect member 103 has a width W3. In some embodiments, the width W3 may be less than about 0.25 cm, less than about 0.4 cm, less than about 0.5 cm, less than about 0.75 cm, less than about 1 cm, less than about 1.5 cm, less than about 2 cm, less than about 2.5 cm, or as per desired application attributes. In the illustrated embodiment of FIG. 1A, the width W3 of the interconnect member 103 is less than the widths W1, W2 of the absorbent pads 102A, 102B. However, in some other embodiments, the width W3 of the interconnect member 103 may be substantially equal to the widths W1, W2 of the absorbent pads 102A, 102B. In some other embodiments, the width W3 of the interconnect member 103 may be greater than the widths W1, W2 of the absorbent pad 102A, 102B. The width W3 may correspond to a normal unstretched width of the interconnect member 103.

In the illustrated embodiment of FIGS. 1A and 1B, a length of the interconnect member 103 is substantially equal to the lengths L1, L2 of the of the absorbent pads 102A, 102B. However, in some other embodiments, the length of the interconnect member 103 may be less than the lengths L1, L2 of the of the absorbent pads 102A, 102B.

As illustrated in FIGS. 1A and 1B, each absorbent pad 102 A, 102B may have curved or rounded edges. The curved edges may prevent any bruises or injures to oral tissues in the oral environment of the patient.

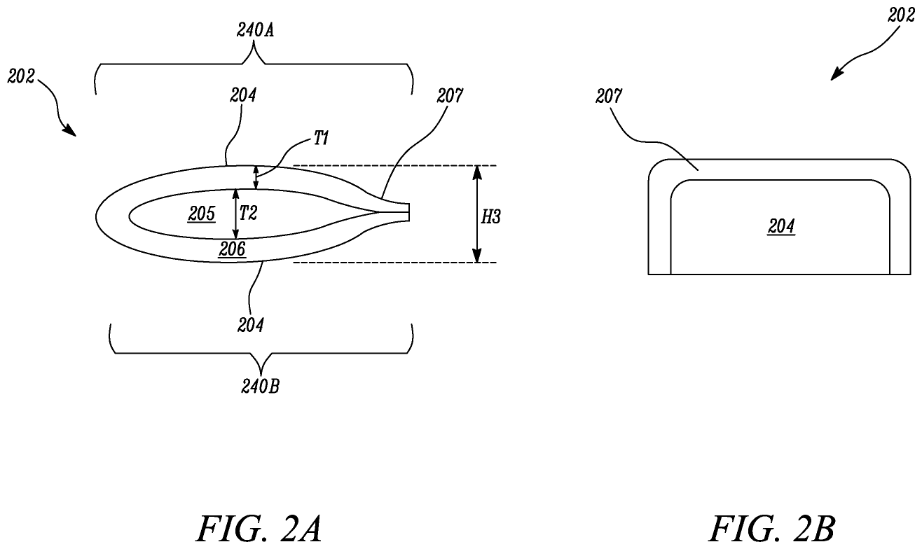

FIGS. 2A and 2B illustrate a schematic cross sectional view and a schematic top view, respectively, of an absorbent pad 202. The absorbent pad 202 may be substantially similar to the absorbent pads 102A, 102B illustrated in FIGS. 1A and 1B. The absorbent pad 202 includes an outer surface 204. In some embodiments, the absorbent article 101, as illustrated in FIGS. 1A and 1B, may include at least two absorbent pads 202. In some embodiments, the interconnect member 103 (shown in FIGS. 1A and 1B) joins the outer surfaces 204 of the at least two absorbent pads 202.

Each absorbent pad 202 includes an outer layer 206. Each absorbent pad 202 further includes an inner layer 205 disposed adjacent to the outer layer 206. The outer layer 206 defines the outer surface 204.

As used herein, the term “layer” refers to a thickness of material that has a relatively consistent formula. A given layer may be combined or connected to other layers. A layer may be either partially or fully continuous as compared to adjacent layers. A given layer may be partially or fully coextensive with the adjacent layers.

As used herein, the term “inner layer” refers to a layer which does not reside on the outer surface 204 of the absorbent pad 202. The inner layer 205 may include a single layer or may be multi-layered.

The outer layer 206 of the absorbent pad 202 is folded upon itself to form a first portion 240A and a second portion 240B connected to the first portion 240A, such that the inner layer 205 is enclosed by the outer layer 206.

The outer layer 206 is made of a liquid permeable material. In some embodiments, the outer layer 206 is hydrophilic. The outer layer 206 may therefore readily allow the liquid to pass to the inner layer 205 disposed adjacent to the outer layer 206. The inner layer 205 includes the absorbent material configured to absorb the liquid. In some embodiments, the outer layer 206 is tear resistant. In such embodiments, the absorbent pad 202 may be placed in the oral environment without tearing or perforating of the outer layer 206 due to contact with the teeth of the patient. However, in some embodiments, on addition of an additive, the outer layer 206 may turn from hydrophilic to hydrophobic. The additive may be added to the outer layer 206 once absorbent pad 202 is saturated with the liquid.

In some embodiments, the outer layer 206 may have an average thickness T1. In some embodiments, the average thickness T1 may be greater than about 0.1 mm, greater than about 0.25 mm, greater than about 0.5 mm, greater than about 0.75 mm, or as per desired application attributes. In some embodiments, the inner layer 205 has a maximum thickness T2. In some embodiments, the maximum thickness T2 may be greater than about 5 mm, greater than about 7.5 mm, greater than about 10 mm, greater than about 12 mm, or as per desired application attributes. In the illustrated embodiment of FIG. 2A, the average thickness T1 of the outer layer 206 is less than the maximum thickness T2 of the inner layer 205. In some embodiments, the average thickness T1 of the outer layer 206 may be less than the maximum thickness T2 of the inner layer 205. However, in some other embodiments, the average thickness T1 of the outer layer 206 may be substantially equal to the thickness T2 of the inner layer 205.

In some embodiments, each absorbent pad 202 has a maximum height H3. In some embodiments, the maximum height H3 may be greater than about 2 mm, greater than about 4 mm, greater than about 5 mm, greater than about 6 mm, greater than about 8 mm, greater than about 10 mm, greater than about 12 mm, or as per desired application attributes. In some embodiments, the maximum height H3 of the absorbent pad 202 may be substantially similar to one of the heights H1, H2 of the absorbent pads 102A, 102B shown in FIG. 1B. However, in some other embodiments, the maximum height H3 and the heights H1, H2 may be different.

In the illustrated embodiment of FIGS. 2A and 2B, the absorbent pad 202 has a rectangular shape. However, the absorbent pad 202 may have any suitable shape, as per desired application attributes.

Each absorbent pad 202 further includes a seal 207 formed between the first portion 240A and the second portion 240B. The seal 207 may be formed using any sealing method, such as heat sealing, ultrasonic sealing, frictional heating, adhesive bonding, stitching, and so forth.

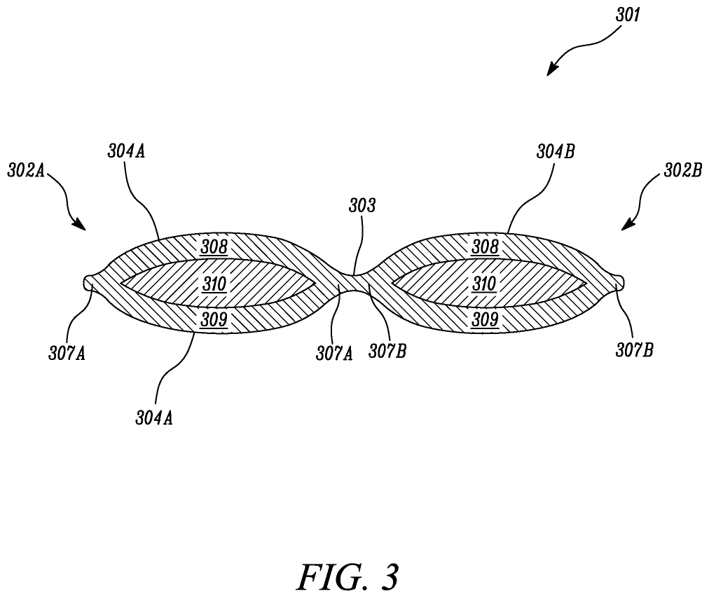

FIG. 3 illustrates a schematic cross-sectional view of an absorbent article 301. The absorbent article 301 may be substantially similar to the absorbent article 101 illustrated in FIGS. 1A and 1B. The absorbent article 301 includes at least two absorbent pads 302A, 302B spaced apart from each other. Each absorbent pad 302A, 302B further includes an outer surface 304A, 304B. Specifically, the absorbent pad 302A includes the outer surface 304A. The absorbent pad 302B includes the outer surface 304B. The absorbent article 301 further includes an interconnect member 303. The interconnect member 303 joins the outer surfaces 304A, 304B of the at least two absorbent pads 302A, 302B.

Each absorbent pad 302A, 302B includes a first layer 308 made of a liquid permeable material. Each absorbent pad 302A, 302B further includes a second layer 309. Each absorbent pad 302A, 302B further includes a third layer 310 disposed between the first layer 308 and the second layer 309. The first layer 308 and the second layer 309 together define the outer surface 304A, 304B. The third layer 310 is enclosed by the first layer 308 and the second layer 309.

In some embodiments, the first layer 308 is hydrophilic. The first layer 308 may therefore readily allow the liquid to pass to the third layer 310 disposed adjacent to the first layer 308. The third layer 310 includes the absorbent material configured to absorb the liquid. The absorbent material may include one or more of superabsorbent particles, superabsorbent fibers, cellulose ethyl sulfonate, carboxymethyl cellulose, sodium polyacrylate, hydrogel particles, hydrogel-based non-woven material, cellulose foam, hydrogel polyurethane, silicone foam, and polyurethane foam. However, in some embodiments, on addition of the additive, the first layer 308 may turn from hydrophilic to hydrophobic. The additive may be added to the outer layer 206 once absorbent pad 202 is saturated with the liquid.

In some embodiments, the first layer 308 is tear resistant. In such embodiments, the absorbent article 301 may be placed in the oral environment without tearing or perforating of the first layer 308 due to contact with the teeth of the patient.

In some embodiments, the second layer 309 is made of a liquid permeable material. The second layer 309 may therefore allow the liquid from the third layer 310 to pass through. In some other embodiments, the second layer 309 is made of a liquid impermeable material. In some embodiments, the second layer 309 is hydrophobic. In such embodiments, the second layer 309 may not allow the liquid from the third layer 310 to pass through and therefore, trap the liquid in the third layer 310. Thus, the second layer 309 may ensure that a flow of the liquid in each absorbent pad 302A, 302B is unidirectional.

Each absorbent pad 302A, 302B includes a seal 307A, 307B formed between the first layer 308 and the second layer 309. Specifically, the absorbent pad 302A includes the seal 307A. The absorbent pad 302A includes the seal 307B. The seals 307A, 307B may be formed using any sealing method, such as heat sealing, ultrasonic sealing, frictional heating, adhesive bonding, stitching, and so forth. In some embodiments, at least a portion of the seals 307A, 307B may join the interconnect member 303 to each of the absorbent pads 302A, 302B.

The interconnect member 303 is stretchable. In some embodiments, the interconnect member 303 is made of an elastomeric material. In some embodiments, the elastomeric material is rubber. In some embodiments, the interconnect member 303 is made of a liquid impermeable material. In some embodiments, the interconnect member 303 includes at least one of polyurethane, styrene block copolymers, polyester elastomers, and elastomeric polyolefins. In some embodiments, the interconnect member 303 is made of a non-woven material.

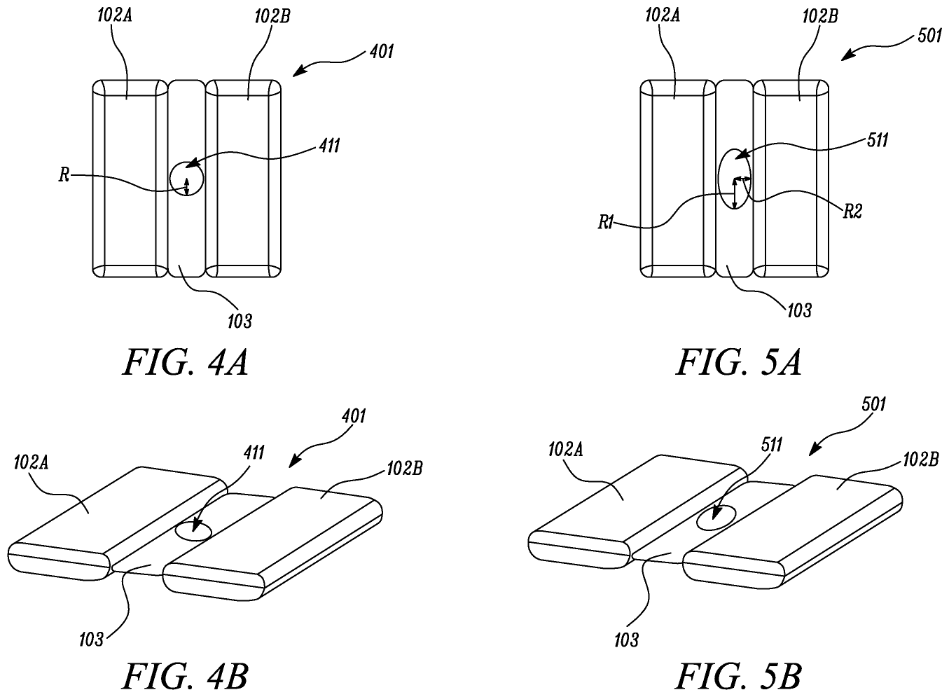

FIGS. 4A and 4B depict a schematic top view and a schematic perspective view, respectively, of an absorbent article 401. The absorbent article 401 is substantially similar to the absorbent article 101 illustrated in FIG. 1A. However, the interconnect member 103 includes one or more through apertures 411. Each of the one or more through apertures 411 is configured to receive at least one tooth.

In the illustrated embodiment of FIGS. 4A and 4B, the interconnect member 103 includes only one through aperture 411. In some embodiments, the through aperture 411 may be configured to receive one tooth. In some embodiments, the through aperture 411 may be configured to receive more than one tooth. In the illustrated embodiment of FIGS. 4A and 4B, the through aperture 411 is substantially circular in shape and has a radius R. In some embodiments, the radius R may be less than about 0.25 cm, less than about 0.5 cm, or as per desired application attributes.

In some other embodiments, multiple through apertures (such as, the through aperture 411) may be present. In some embodiments, each of the multiple through apertures may be configured to receive one tooth. In some embodiments, the multiple through apertures may be similar in size and shape. In some other embodiments, the multiple through apertures may be different in size and shape. The size and shape of the one or more through apertures 411 may vary based on the desired application attributes.

FIGS. 5A and 5B depict a schematic top view and a schematic perspective view, respectively, of an absorbent article 501. The absorbent article 501 is substantially similar to the absorbent article 401 illustrated in FIGS. 4A and 4B. However, the interconnect member 103 includes one or more through apertures including a slit 511. The slit 511 is configured to receive at least one tooth. In some embodiments, the slit 511 may be configured to receive more than one tooth. In the illustrated embodiment of FIGS. 5A and 5B, the slit 511 is substantially oval in shape and has a maximum radius R1 and a minimum radius R2. In some embodiments, the maximum radius R1 may be less than about 0.1 cm, less than about 0.25 cm, less than about 0.5 cm, less than about 0.75 cm, or as per desired application attributes. In some embodiments, the minimum radius R2 may be less than about 0.1 cm, less than about 0.25 cm, less than about 0.5 cm, or as per desired application attributes.

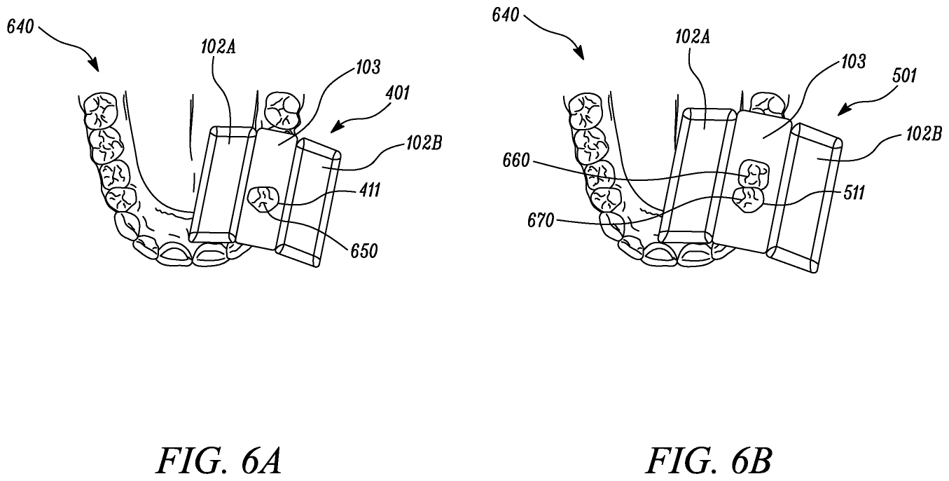

FIG. 6A depicts a schematic view of a lower jaw 640 of the patient and the absorbent article 401 in use. The liquid, such as blood, saliva and/or water, may be produced and accumulated in the lower jaw 640. In some other embodiments, the absorbent article 401 may be placed in an upper jaw (not shown) of the patient. The interconnect member 103 includes the through aperture 411 as shown in FIG. 4A. When in use, the interconnect member 103 is stretched and placed over at least one tooth 650 to be worked on by the dental clinician. Specifically, the interconnect member 103 is stretched and placed over the at least one tooth 650 such that the through aperture 411 may surround and secure the at least one tooth 650. In some embodiments, the through aperture 411 may take a shape of the at least one tooth 650. In some embodiments, the interconnect member 103 is stretched and placed over the at least one tooth 650 such that the multiple through apertures (not shown) surround and secure a corresponding tooth. Specifically, the at least one tooth 650 may project through the through aperture 411 providing a working area visibility and accessibility to the dental clinician.

The absorbent pads 102A, 102B may be placed on either side of the at least one tooth 650. This placement of the absorbent pads 102A, 102B and the interconnect member 103 of the absorbent article 401 may isolate the at least one tooth 650 and may ensure that the liquid is absorbed from the oral environment efficiently and without discomfort to the patient. The absorbent article 401 may further prevent the liquid from entering a throat of the patient. The absorbent article 401 may thereby keep the oral environment dry and provide the working area visibility and accessibility to the dental clinician.

In the illustrated embodiments of FIG. 6A, the absorbent article 401 is shown. However, in other embodiments, the absorbent articles 101 and 301 (shown in FIGS. 1A and 3, respectively) may also be used in a similar manner. When using the absorbent articles 101 and 301, the dental clinician may penetrate or puncture the interconnect member 103, 303 to form the through aperture 411 before stretching and placing the interconnect member 103, 303 over the at least one tooth 650. In some embodiments, the dental clinician may penetrate or puncture the interconnect member 103, 303 to form the multiple through apertures to receive the corresponding tooth.

FIG. 6B depicts a schematic view of the lower jaw 640 of the patient and the absorbent article 501 in use. In some embodiments, the absorbent article 501 may be placed in the upper jaw (not shown) of the patient. The interconnect member 103 includes the slit 511, as shown in FIG. 5A. When in use, the interconnect member 103 is stretched and placed over at least one tooth to be worked on by the dental clinician. Specifically, the interconnect member 103 is stretched and placed over the at least one tooth such that the slit 511 may surround and secure the at least one tooth. In the illustrated embodiment of FIG. 6B, the slit 511 surrounds and secures two teeth 660, 670. In some embodiments, the slit 511 may take a shape of the teeth 660, 670. However, in some other embodiments, the slit 511 may surround and secure any one of the teeth 660, 670. Specifically, the teeth 660, 670 may project through the slit 511 providing the working area visibility and accessibility to the dental clinician.

The absorbent pads 102A, 102B are placed on the either side of the teeth 660, 670. This placement of the absorbent pads 102A, 102B and the interconnect member 103 of the absorbent article 501 may isolate the teeth 660, 670 and may ensure that the liquid is absorbed from the oral environment efficiently and without discomfort to the patient. The absorbent article 501 may further prevent the liquid from entering the throat of the patient. The absorbent article 501 may thereby keep the oral environment dry and provide the working area visibility and accessibility to the dental clinician.

In the illustrated embodiments of FIG. 6B, the absorbent article 501 is shown. However, in other embodiments, the absorbent articles 101 and 301 may also be used in a similar manner. When using the absorbent articles 101 and 301, the dental clinician may penetrate or puncture the interconnect member 103, 303 to form the slit 511 before stretching and placing the interconnect member 103, 303 over the teeth 660, 670.

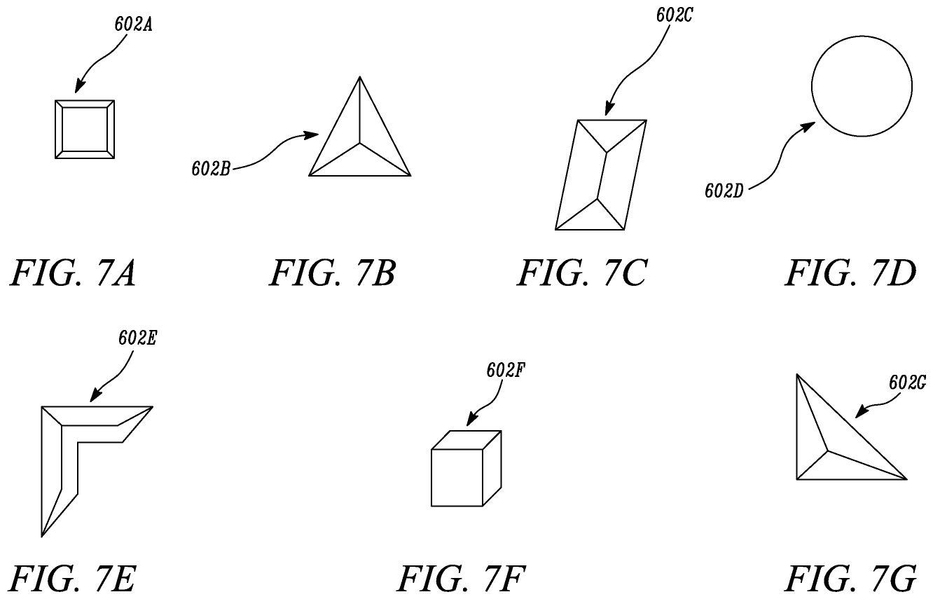

FIGS. 7A to 7G illustrate schematic views of various absorbent pads having different shapes. Specifically, FIGS. 7A to 7G depict respective absorbent pads 602A, 602B, 602C, 602D, 602E, 602F, 602G. The absorbent pad 602A has a substantially cuboidal shape. The absorbent pad 602B has a pyramidal shape. The absorbent pad 602C has a prismatic shape. The absorbent pad 602D has a spherical shape. The absorbent pad 602E has a substantially L-shape. The absorbent pad 602F has a cubical shape. The absorbent pad 602G a triangular pyramidal shape. As apparent from FIGS. 7A to 7G, the absorbent pads 602A, 602B, 602C, 602D, 602E, 602F, 602G may be designed in any shape to fit into different parts of the mouth of the patient without causing discomfort. In some embodiments, the absorbent pads 602A, 602B, 602C, 602D, 602E, 602F, 602G may be filled with the absorbent material through an injection molding process.

Similarly, the absorbent pads 602A, 602B, 602C, 602D, 602E, 602F, 602G may have any size depending upon a size of the mouth of the patient. For example, if the patient is a child, a size of the absorbent pad 602A, 602B, 602C, 602D, 602E, 602F, 602G may be relatively smaller.

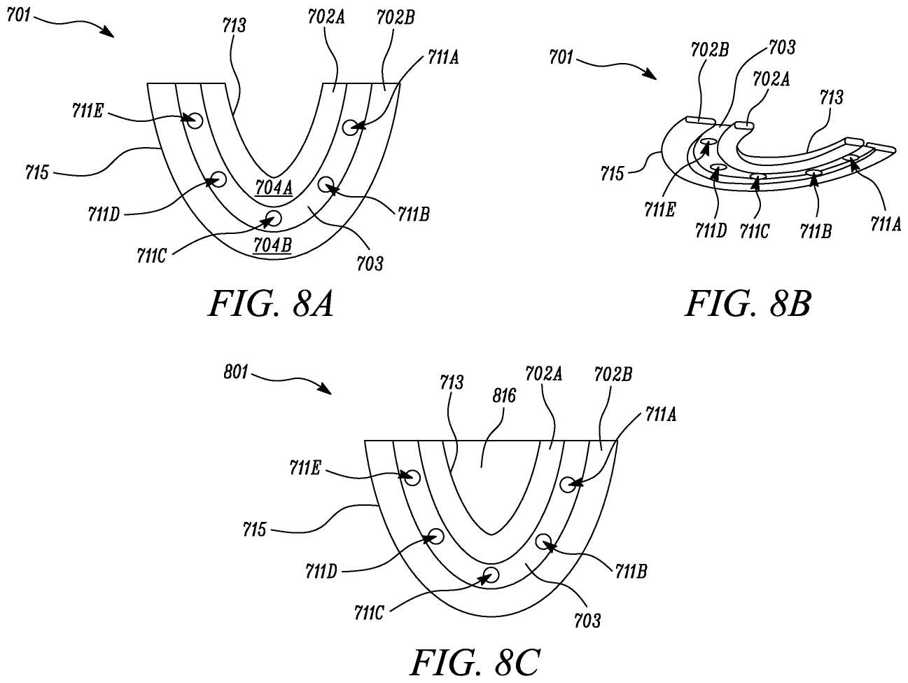

FIGS. 8A and 8B depict a schematic top and a schematic perspective view, respectively of an absorbent article 701. The absorbent article 701 is substantially similar to the absorbent article 101 illustrated in FIGS. 1A and 1B. The absorbent article 701 includes at least two absorbent pads 702A, 702B spaced apart from each other. However, as illustrated in FIGS. 8A and 8B, each absorbent pad 702A, 702B is arc-shaped, such that a first absorbent pad 702A of the at least two absorbent pads 702A, 702B forms an inner arc 713 and a second absorbent pad 702B of the at least two absorbent pads 702A, 702B forms an outer arc 715. The inner arc 713 and the outer arc 715 may correspond to a shape of the lower jaw 640 (shown in FIGS. 6A and 6B) of the patient. Therefore, the absorbent article 701 may fit in the mouth of the patient with ease and without causing discomfort to the patient.

Each absorbent pad 702A, 702B further includes an outer surface 704A, 704B. An interconnect member 703 joins the outer surfaces 704A, 704B of the at least two absorbent pads 702A, 702B. In some embodiments, the interconnect member 703 includes one or more through apertures 711A, 711B, 711C, 711D, 711E (collectively, one or more through apertures 711). Each of the one or more through apertures 711 is configured to receive at least one tooth. In the illustrated embodiment of FIGS. 8A and 8B, the interconnect member 703 includes five through apertures 711. However, in some other embodiments, the interconnect member 703 may include more than five through apertures 711. In some other embodiments, the interconnect member 703 may include less than five through apertures 711. In some other embodiments, the interconnect member 703 may include one through aperture 711.

The first absorbent pad 702A and the second absorbent pad 702B of the absorbent article 701 may cover either side of the at least one tooth while exposing only the at least one tooth to the dental clinician. The absorbent article 701 may thereby keep the oral environment dry and may further provide the working area visibility and accessibility to the dental clinician.

FIG. 8C illustrates a schematic top view of an absorbent article 801. The absorbent article 801 is substantially similar to the absorbent article 701. However, the absorbent article 801 further includes an inner member 816 attached to the first absorbent pad 702A at least partially along the inner arc 713. In some embodiments, the inner member 816 is stretchable. In some embodiments, the inner member 816 is made of a liquid impermeable material. In some embodiments, the interconnect member 703 and the inner member 816 of the absorbent article 701 are made of a similar material. The inner member 816 may be placed over a tongue of the patient. The inner member 816 may cover the tongue of the patient and may protect the patient from sharp tools used by the dental clinician. The inner member 816 may further restrict the flow of the liquid to the tongue of the patient. The inner member 816 may further help to keep the tongue of the patient away from the at least one tooth.

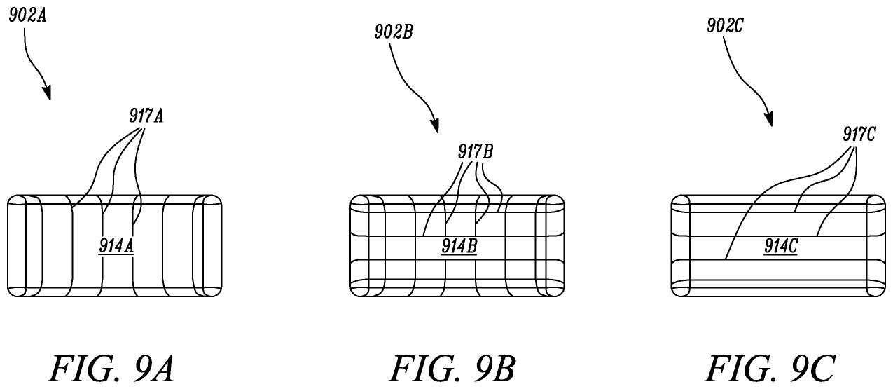

FIGS. 9A, 9B and 9C illustrate absorbent pads 902A, 902B, 902C, respectively. The absorbent pads 902A, 902B, 902C are substantially similar to the absorbent pad 102A of FIG. 1A. The absorbent pads 902A, 902B, 902C include outer surfaces 914A, 914B, 914C, respectively. However, each absorbent pad 902A, 902B, 902C further includes a wire mesh 917A, 917B, 917C disposed on the outer surface 914A, 914B, 914C. Specifically, the absorbent pad 902A incudes the wire mesh 917A disposed on the outer surface 914A. The absorbent pad 902B incudes the wire mesh 917B disposed on the outer surface 914B. Further, the absorbent pad 902C incudes the wire mesh 917C disposed on the outer surface 914C. In some embodiments, each of the wire meshes 917A, 917B, 917C may include ductile wires. The wire meshes 917A, 917B, 917C may help to conform the respective absorbent pads 902A, 902B, 902C to a desired shape to fit in the mouth of the patient. In other words, the wire meshes 917A, 917B, 917C may help to deform and alter a shape of the respective absorbent pads 902A, 902B, 902C to conform to different inner sizes and shapes of the mouth. The absorbent pads 902A, 902B, 902C including the wire meshes 917A, 917B, 917C may provide a good coverage in an area where the liquid, such as blood, saliva and/or water, is produced and accumulated, and may therefore provide an efficient absorption of the liquid from the oral environment. In some other embodiments, the wire meshes 917A, 917B, 917C may be embedded in the respective absorbent pads 902A, 902B, 902C. For example, the wire meshes 917A, 917B, 917C may be embedded the third layer 310 (shown in FIG. 3). In such embodiments, the wire meshes 917A, 917B, 917C may hold the absorbent material in place. In some other embodiments, the wire meshes 917A, 917B, 917C may be embedded in the first layer 308 (shown in FIG. 3) and/or the second layer 309 (shown in FIG. 3).

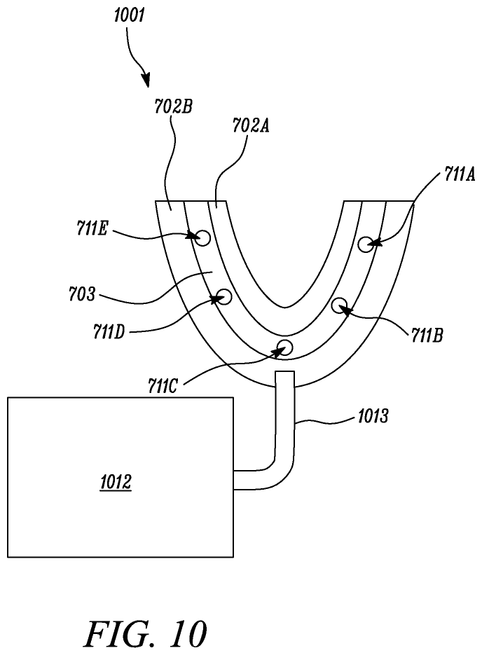

FIG. 10 illustrates a schematic view of an absorbent article 1001. The absorbent article 1001 is substantially similar to the absorbent article 701 of FIG. 8A. However, at least one of the two absorbent pads 702A, 702B includes a tube 1013 in fluid communication with the absorbent material. The tube 1013 is configured to be attached to a suction source 1012. The suction source 1012 may create a negative pressure inside the tube 1013. In some embodiments, the tube 1013 is flexible and may easily bend and adapt to other shapes. The suction source 1012 may provide active suction to remove the excess liquid from the at least one of the two absorbent pads 702A, 702B once the at least one of the two absorbent pads 702A, 702B is saturated. In some embodiments, the suction source 1012 may be placed away from the absorbent article 701.

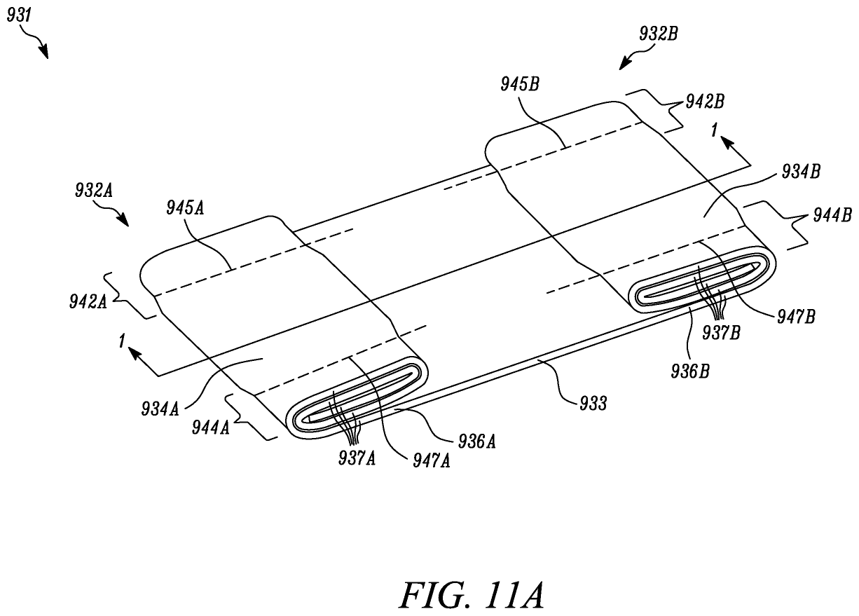

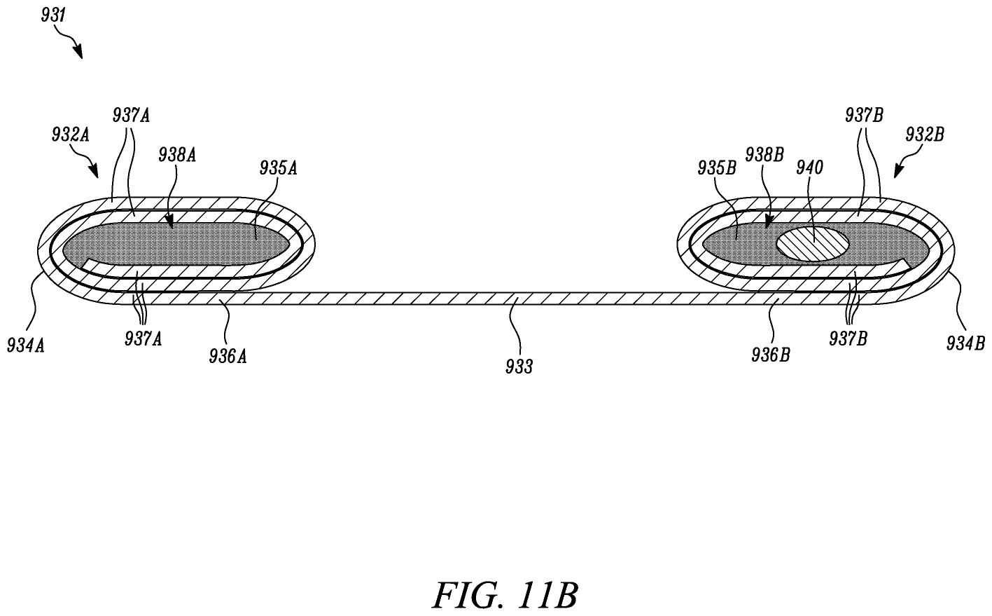

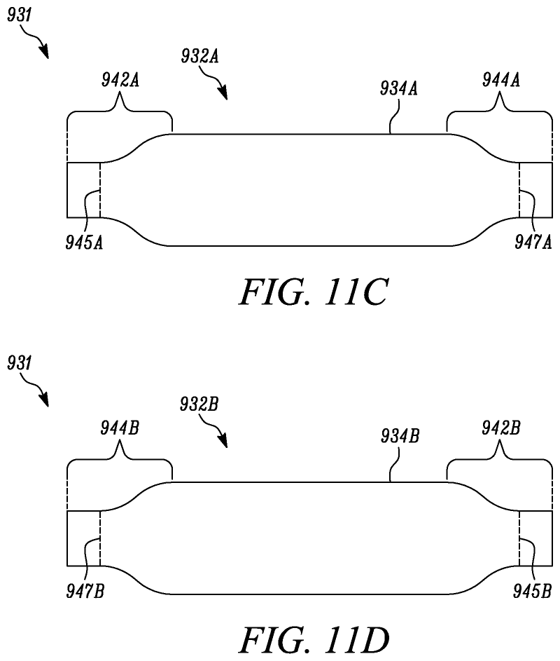

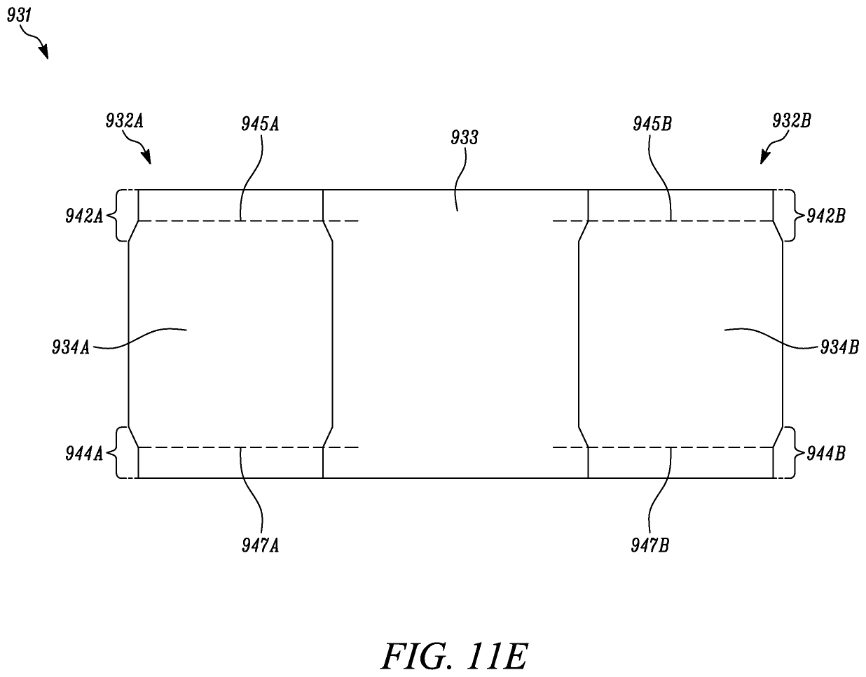

FIGS. 11A-11E illustrate an absorbent article 931 according to another embodiment of the present disclosure. Specifically, FIG. 11A illustrates a schematic perspective view of the absorbent article 931, FIG. 11B illustrates a schematic sectional view of the absorbent article 931 taken along a line 1-1 of FIG. 11A, FIG. 11C illustrates a schematic front view of the absorbent article 931, FIG. 11D illustrates a schematic rear view of the absorbent article 931, and FIG. 11E illustrates a schematic top view of the absorbent article 931.

Referring to FIGS. 11A-11E, the absorbent article 931 includes at least two absorbent pads 932A, 932B spaced apart from each other. Each absorbent pad 932A, 932B includes an outer surface 934A, 934B. Specifically, the absorbent pad 932A includes the outer surface 934A, and the absorbent pad 932B includes the outer surface 934B.

Each absorbent pad 932A, 932B includes an absorbent material 935A, 935B (shown in FIG. 11B). In other words, the absorbent material 935A, 935B is disposed within each absorbent pad 932A, 932B. Specifically, the absorbent pad 932A includes the absorbent material 935A, and the absorbent pad 932B includes the absorbent material 935B. The absorbent material 935A, 935B is configured to absorb a liquid. In some embodiments, the liquid includes at least one of saliva, water, and blood.

In some embodiments, the absorbent material 935A, 935B is made of one or more superabsorbent particles, superabsorbent fibers, cellulose ethyl sulfonate, carboxymethyl cellulose, sodium polyacrylate, hydrogel particles, hydrogel-based non-woven material, cellulose foam, hydrogel polyurethane, silicone foam, and polyurethane foam. The absorbent material 935A may be similar to, or different from, the absorbent material 935B.

Furthermore, the absorbent material 935A, 935B may have a suitable absorption rate, as per desired application attributes. In some embodiments, the absorption of the liquid may not significantly increase a size or a volume of the absorbent pads 932A, 932B. However, in some other embodiments, the absorption of the liquid may increase the size or the volume of the absorbent pads 932A, 932B.

The absorbent article 931 further includes an interconnect member 933. The interconnect member 933 joins the outer surfaces 934A, 934B of the at least two absorbent pads 932A, 932B. Further, the interconnect member 933 is stretchable.

In some embodiments, each absorbent pad 932A, 932B further includes a film 936A, 936B. Specifically, in some embodiments, the absorbent pad 932A includes the film 936A, and the absorbent pad 932B includes the film 936B. The film 936A, 936B is made of a liquid permeable material. The film 936A, 936B may allow a liquid (e.g., saliva, water, and blood) to pass therethrough.

In some embodiments, the film 936A, 936B includes at least one of polyurethane, styrene block copolymers, polyester elastomers, elastomeric polyolefins, and a non-woven material. In some embodiments, the film 936A, 936B is tear resistant. In some embodiments, the film 936A, 936B is hydrophilic. The film 936A may be similar to, or different from, the film 936B.

As shown in FIGS. 11A and 11B, in some embodiments, the film 936A, 936B is folded upon itself to form three or more folded layers 937A, 937B. Specifically, in some embodiments, the film 936A is folded upon itself to form the three or more folded layers 937A, and the film 936B is folded upon itself to form the three or more folded layers 937B. The film 936A, 936B may be folded upon itself to form three, more than three, more than four, more than five, more than six, more than seven, or more than eight folded layers 937A, 937B.

The film 936A, 936B may at least partially form the outer surface 934A, 934B of each absorbent pad 932A, 932B. Specifically, the film 936A may at least partially form the outer surface 934A of the absorbent pad 932A, and the film 936B may at least partially form the outer surface 934B of the absorbent pad 932B.

In the illustrated embodiment of FIG. 11B, the film 936A, 936B further forms a hollow core 938A, 938B. Specifically, in the illustrated embodiment of FIG. 11B, the film 936A forms the hollow core 938A, and the film 936B forms the hollow core 938B. Furthermore, in the illustrated embodiment of FIG. 11B, the absorbent material 935A, 935B is disposed within the hollow core 938A, 938B. In some embodiments, the absorbent material 935A, 935B disposed within the hollow core 938A, 938B may be in a powdered form.

Moreover, in the illustrated embodiment of FIG. 11B, at least one absorbent pad 932B further includes a spacer material 940 disposed within the hollow core 938B of the at least one absorbent pad 932B. In some embodiments, the spacer material 940 is configured to increase a radial stiffness of the at least one absorbent pad 932B. The spacer material 940 may have any suitable shape and dimensions to increase the radial stiffness of the at least one absorbent pad 932B. The spacer material 940 may occupy, for example, greater than 30%, greater than 40%, greater than 50%, greater than 60%, or greater than 70% of a volume of the hollow core 938B. In some examples, the spacer material 940 may include a stiff fabric.

In some embodiments, each absorbent pad 932A, 932B may include the spacer material 940 disposed within the hollow core 938A, 938B, and the spacer material 940 may increase a radial stiffness of each absorbent pad 932A, 932B. However, it may be noted that the spacer material 940 is optional and may be omitted.

Further, the film 936A, 936B may fully enclose the absorbent material 935A, 935B by any suitable means (e.g., by a sealing member), such that the absorbent material 935A, 935B is not exposed and is contained within each absorbent pad 932A, 932B. In some examples, the film 936A, 936B may further fully enclose the spacer material 940.

As shown in FIGS. 11A and 11C-11E, in some embodiments, the film 936A, 936B of each absorbent pad 932A, 932B includes a first tapered portion 942A, 942B and a second tapered portion 944A, 944B opposite to the first tapered portion 942A, 942B, such that the film 936A, 936B fully encloses the absorbent material 935A, 935B (shown in FIG. 11B). The first tapered portion 942A, 942B and the second tapered portion 944A, 944B may be formed by pinching and compressing opposite portions of the film 936A, 936B.

In some embodiments, the film 936A of the absorbent pad 932A includes the first tapered portion 942A and the second tapered portion 944A opposite to the first tapered portion 942A, such that the film 936A fully encloses the absorbent material 935A (shown in FIG. 11B). Further, in some embodiments, the film 936B of the absorbent pad 932B includes the first tapered portion 942B and the second tapered portion 944B opposite to the first tapered portion 942B, such that the film 936B fully encloses the absorbent material 935B (shown in FIG. 11B). In some cases, each of the first tapered portion 942A, 942B and the second tapered portion 944A, 944B may have a portion that is relatively planar.

In some embodiments, each absorbent pad 932A, 932B further includes at least a first seal 945A, 945B (shown by dashed lines in FIGS. 11A and 11C-11E) configured to seal the film 936A, 936B at the first tapered portion 942A, 942B and at least a second seal 947A, 947B (shown by dashed lines in FIGS. 11A and 11C-11E) configured to seal the film 936A, 936B at the second tapered portion 944A, 944B. Specifically, in the illustrated embodiment of FIGS. 11A and 11C-11E, the absorbent pad 932A includes the first seal 945A configured to seal the film 936A at the first tapered portion 942A and the second seal 947A configured to seal the film 936A at the second tapered portion 944A. Furthermore, in the illustrated embodiment of FIGS. 11A and 11C-11E, the absorbent pad 932B includes the first seal 945B configured to seal the film 936B at the first tapered portion 942B and the second seal 947B configured to seal the film 936B at the second tapered portion 944B. The first seal 945A, 945B and the second seal 947A, 947B may prevent dislodgement of the absorbent material 935A, 935B from the hollow core 938A, 938B of the film 936A, 936B.

The first seal 945A, 945B and the second seal 947A, 947B may be formed by any suitable sealing method, such as heat sealing, ultrasonic sealing, frictional heating, adhesive bonding, stitching, and so forth. For example, the first seal 945A, 945B and the second seal 947A, 947B may be ultrasonic welds. In some examples, the first seal 945A, 945B and the second seal 947A, 947B may partially extend on the interconnect member 933.

Moreover, in some embodiments, the interconnect member 933 and the film 936A, 936B of each absorbent pad 932A, 932B form a single integral film member. For example, the film 936A may be a first portion of the single integral film member, the interconnect member 933 may be a second portion of the single integral film member, and the film 936B may be a third portion of the single integral film member. In other words, in some embodiments, the film 936A and the film 936B may be integral. Moreover, in such embodiments, the absorbent article 931 may be formed by folding the single integral film member including the films 936A, 936B at opposite ends to form the at least two absorbent pads 932A, 932B, such that the single integral film member further forms the interconnect member 933 between the at least two absorbent pads 932A, 932B.

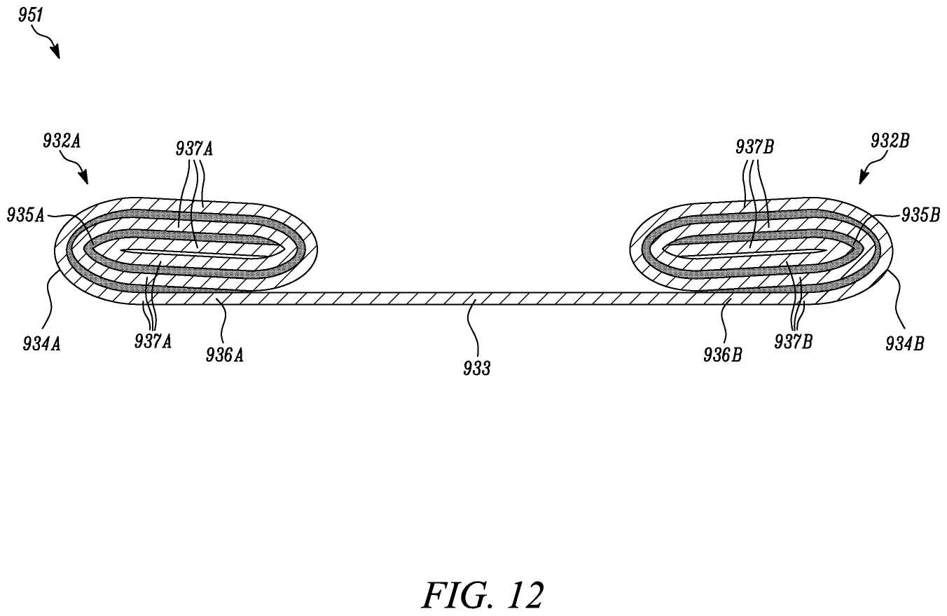

FIG. 12 illustrates a schematic sectional side view of an absorbent article 951 according to another embodiment of the present disclosure. The absorbent article 951 is similar to the absorbent article 931 of FIGS. 11A-11E, with like elements designated by like references numerals. However, the absorbent article 951 has a different disposition of the absorbent material 935A, 935B as compared to the absorbent article 931 of FIGS. 11A-11E.

In the illustrated embodiment of FIG. 12, the absorbent material 935A, 935B is disposed between at least two adjacent folded layers 937A, 937B from the three or more folded layers 937A, 937B. Specifically, in the illustrated embodiment of FIG. 12, the absorbent material 935A is disposed between at least two adjacent folded layers 937A from the three or more folded layers 937A, and the absorbent material 935B is disposed between at least two adjacent folded layers 937B from the three or more folded layers 937B. In some embodiments, the absorbent material 935A, 935B disposed between at least two adjacent folded layers 937A, 937B may be in a fibrous form.

In such embodiments, one or more seals may be provided (in addition to the first seal 945A, 945B and the second seal 947A, 947B of FIGS. 11A and 11C-11E) to ensure that the absorbent material 935A, 935B is fully enclosed by the film 936A, 936B. Furthermore, in such embodiments, the film 936A, 936B may or may not form a hollow core (similar to the hollow core 938A, 938B shown in FIG. 11B), depending upon a type of fold and a fold tightness of the film 936A, 936B. In embodiments where the hollow core is formed, the absorbent material 935A, 935B may be further disposed within the hollow core. Further, in some cases, a spacer material (similar to the spacer material 940 shown in FIG. 11B) may be optionally disposed within the hollow core.

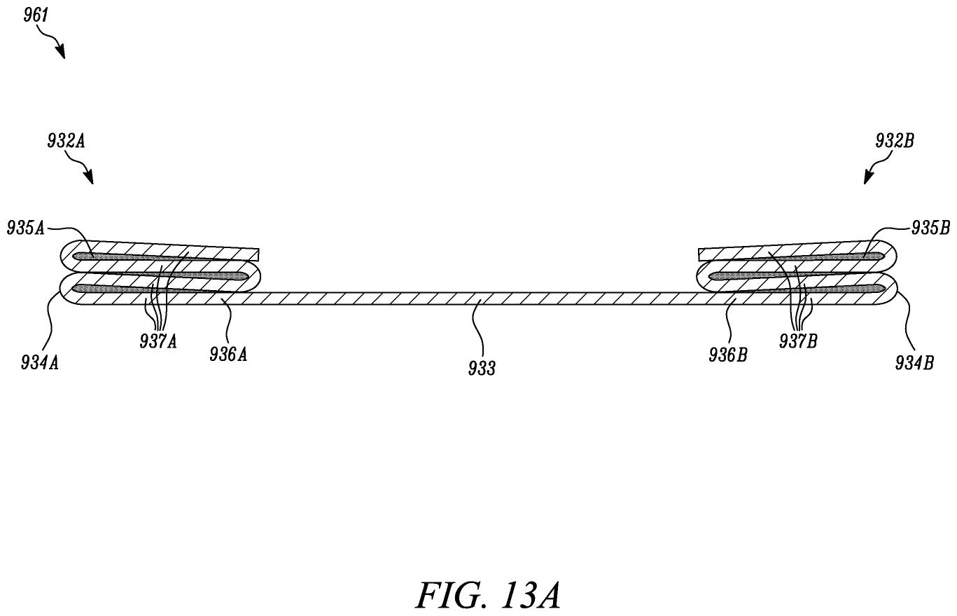

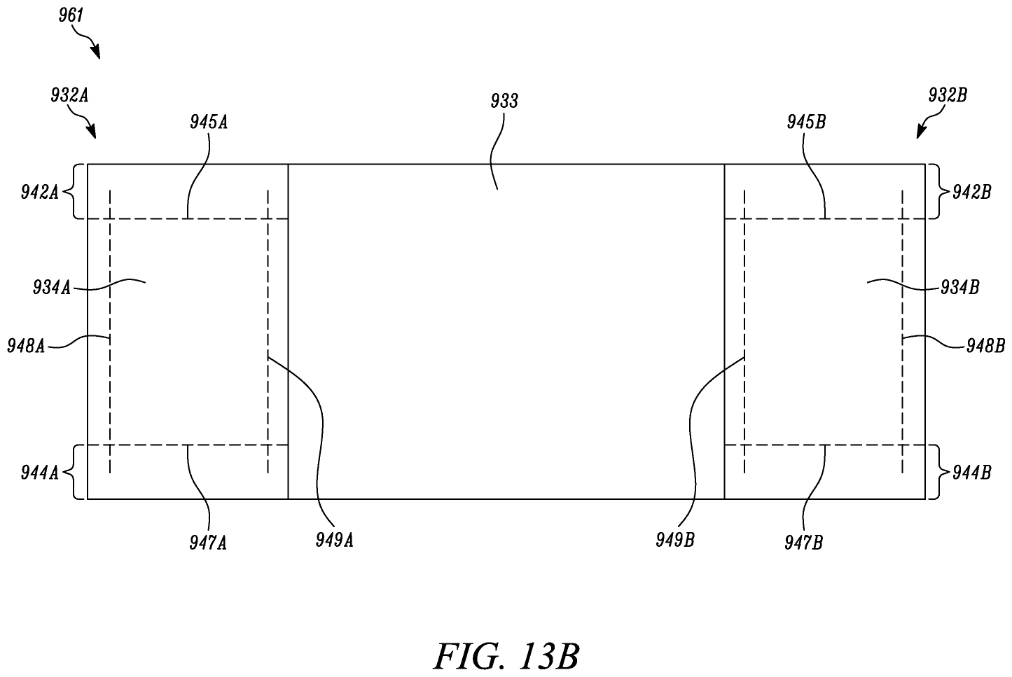

FIGS. 13A and 13B illustrate an absorbent article 961 according to another embodiment of the present disclosure. Specifically, FIG. 13A illustrates a schematic sectional side view of the absorbent article 961, and FIG. 13B illustrates a schematic top view of the absorbent article 961. The absorbent article 961 is similar to the absorbent article 951 of FIG. 12, with like elements designated by like references numerals. However, the absorbent article 961 has a different configuration of the film 936A, 936B as compared to the absorbent article 951 of FIG. 12.

Specifically, in the illustrated embodiment of FIG. 13A, the film 936A, 936B is folded upon itself in a fan fold configuration (also referred to as an accordion or a z-fold configuration) to form the three or more folded layers 937A, 937B. Further, in the illustrated embodiment of FIG. 13A, the absorbent material 935A, 935B is disposed between at least two adjacent folded layers 937A, 937B from the three or more folded layers 937B, 937B.

As shown in FIG. 13B, in some embodiments, each absorbent pad 932A, 932B may further include a third seal 948A, 948B extending at least between the first seal 945A, 945B and the second seal 947A, 947B, and a fourth seal 949A, 949B opposite to the third seal 948A, 948B and extending at least between the first seal 945A, 945B and the second seal 947A, 947B. The third seal 948A, 948B and the fourth seal 949A, 949B may ensure that the film 936A, 936B fully encloses the absorbent material 935A, 935B in the accordion configuration of the film 936A, 936B.

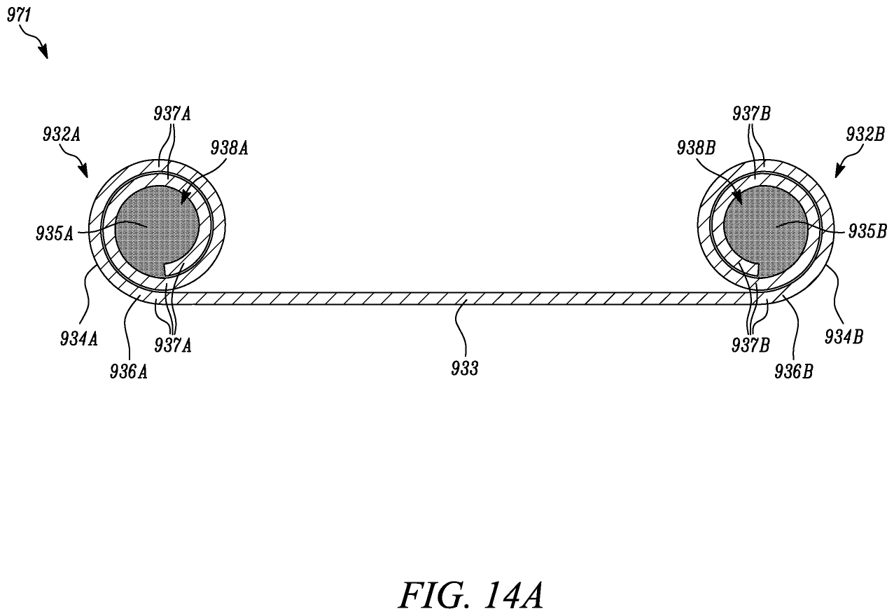

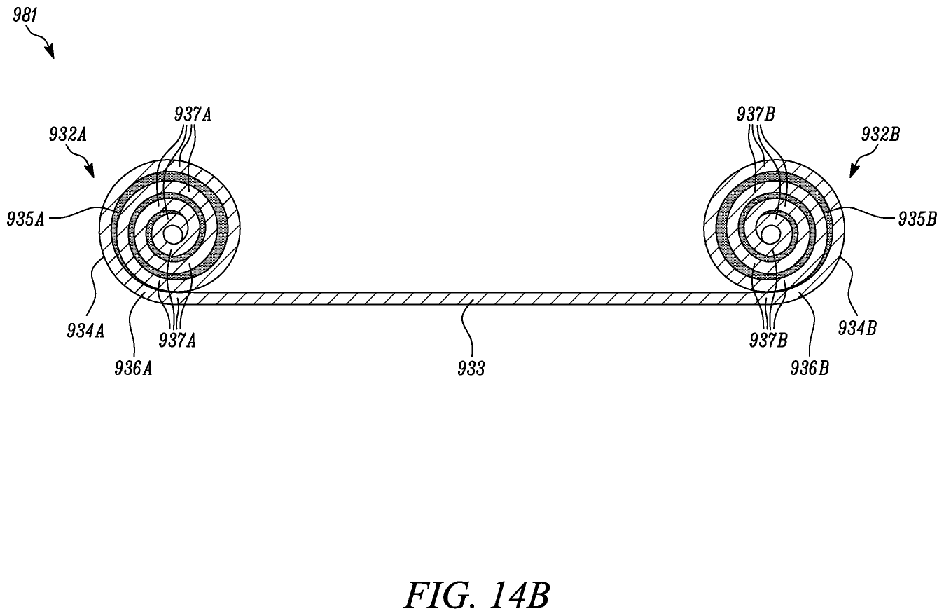

FIG. 14A illustrates a schematic sectional side view of an absorbent article 971 according to another embodiment of the present disclosure. The absorbent article 971 is substantially similar to the absorbent article 931 of FIGS. 11A-11E, with like elements designated by like references numerals. However, the absorbent article 971 has a different configuration of the film 936A, 936B as compared to the absorbent article 931 of FIGS. 11A-11E.

Specifically, in the illustrated embodiment of FIG. 14A, the film 936A, 936B is folded upon itself in a spiral configuration to form the three or more folded layers 937A, 937B. The film 936A, 936B may be folded upon itself in the spiral configuration via any suitable mechanical rotary device. The three or more folded layers 937A, 937B may be concentric or nonconcentric in the spiral configuration. Further, in the illustrated embodiment of FIG. 13A, the film 936A, 936B further forms the hollow core 938A, 938B, and the absorbent material 935A, 935B is disposed within the hollow core 938A, 938B. Specifically, the film 936A forms the hollow core 938A that receives the absorbent material 935A therein. The film 936B forms the hollow core 938A that receives the absorbent material 935B therein.

FIG. 14B illustrates a schematic sectional side view of an absorbent article 981 according to another embodiment of the present disclosure. The absorbent article 981 is similar to the absorbent article 951 of FIG. 12, with like elements designated by like references numerals. However, the absorbent article 981 has a different configuration of the film 936A, 936B as compared to the absorbent article 951 of FIG. 12.

Specifically, in the illustrated embodiment of FIG. 14B, the film 936A, 936B is folded upon itself in a spiral configuration to form the three or more folded layers 937A, 937B. The film 936A, 936B may be folded upon itself in the spiral configuration via any suitable mechanical rotary device. Further, in the illustrated embodiment of FIG. 14B, the absorbent material 935A is disposed between at least two adjacent folded layers 937A from the three or more folded layers 937A, and the absorbent material 935B is disposed between at least two adjacent folded layers 937B from the three or more folded layers 937B. In such embodiments, one or more seals may be provided (in addition to the first seal 945A, 945B and the second seal 947A, 947B of FIGS. 11C-11E) to ensure that the absorbent material 935A, 935B is fully enclosed by the film 936A, 936B.

Furthermore, in such embodiments, the film 936A, 936B may or may not form a hollow core (similar to the hollow core 938A, 938B shown in FIG. 11B), depending upon a fold tightness of the film 936A, 936B. In embodiments where the hollow core is formed, the absorbent material 935A, 935B may be further disposed within the hollow core. Further, in some cases, a spacer material (similar to the spacer material 940 shown in FIG. 11B) may be optionally disposed within the hollow core.

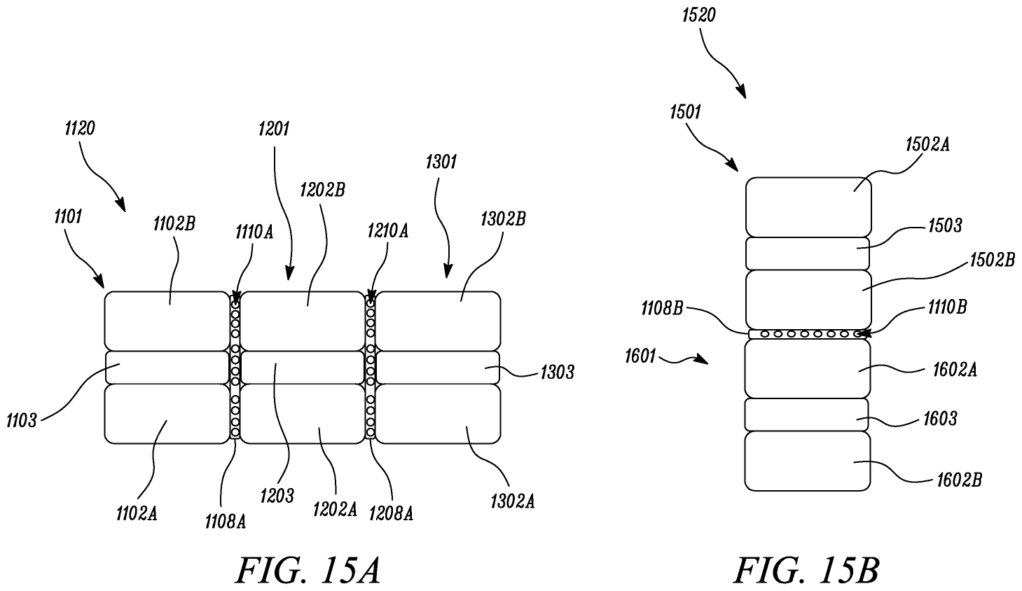

FIG. 15A illustrates a schematic top view of an absorbent system 1120. The absorbent system 1120 includes a plurality of absorbent articles 1101, 1201, 1301. Each of the plurality of absorbent articles 1101, 1201, 1301 is substantially similar to the absorbent article 101, shown in FIG. 1A. In the illustrated embodiment of FIG. 15A, three absorbent articles 1101, 1201, 1301 are shown. However, in some other embodiments, the plurality of absorbent articles 1101, 1201, 1301 may include more than three absorbent articles 1101, 1201, 1301.

The absorbent system 1120 further includes at least one line of weakness 1108A, 1208A. In the illustrated embodiment of FIG. 15A, two lines of weakness 1108A, 1208A are shown. However, in some other embodiments, the at least one line of weakness 1108A, 1208A may include more than two lines of weakness 1108A, 1208A. The line of weakness 1108A joins two adjacent absorbent articles 1101, 1201 of the plurality of absorbent articles 1101, 1201, 1301. The line of weakness 1208A joins two adjacent absorbent articles 1201, 1301 of the plurality of absorbent articles 1101, 1201, 1301. In some embodiments, the at least one line of weakness 1108A, 1208A includes at least one of a cut, a perforation, and a score. In the illustrated embodiment of FIG. 15A, the line of weakness 1108A includes perforations 1110A and the line of weakness 1208A includes perforations 1210A.

As illustrated in FIG. 15A, the at least one line of weakness 1108A joins at least two absorbent pads 1102A, 1102B and interconnect member 1103 of the absorbent article 1101 to at least two absorbent pads 1202A, 1202B and interconnect member 1203 of the adjacent absorbent article 1201. Similarly, the at least one line of weakness 1208A joins the at least two absorbent pads 1202A, 1202B and the interconnect member 1203 of the absorbent article 1201 to at least two absorbent pads 1302A, 1302B and interconnect member 1303 of the adjacent absorbent article 1301.

In some embodiments, the plurality of absorbent articles 1101, 1201, 1301 of the absorbent system 1120 forms a reel (not shown). The lines of weakness 1108A, 1208B may help to detach the plurality of absorbent articles 1101, 1201, 1301 from each other. In some embodiments, the plurality of absorbent articles 1101, 1201, 1301 may be torn along the lines of weakness 1108A, 1208A.

FIG. 15B illustrates a schematic top view of an absorbent system 1520. The absorbent system 1520 includes a plurality of absorbent articles 1501, 1601. Each of the plurality of absorbent articles 1501, 1601 is substantially similar to the absorbent article 101, shown in FIG. 1A. The absorbent articles 1501 includes at least two absorbent pads 1502A, 1502B spaced apart from each other and an interconnect member 1503. The absorbent article 1601 includes at least two absorbent pads 1602A, 1602B spaced apart from each other and an interconnect member 1603. The absorbent system 1520 further includes at least one line of weakness 1108B. The line of weakness 1108B joins two adjacent absorbent articles 1501, 1601 of the plurality of absorbent articles 1501, 1601. Specifically, the at least one line of weakness 1108B joins one absorbent pad 1502B of the at least two absorbent pads 1502A, 1502B of one absorbent article 1501 to one absorbent pad 1602A of the at least two absorbent pads 1602A, 1602B of the adjacent absorbent article 1601. In other words, the line of weakness 1108A joins the absorbent pad 1502B of the absorbent article 1501 to the absorbent pad 1602A of the adjacent absorbent article 1601. In some embodiments, the at least one line of weakness 1108B includes at least one of a cut, a perforation, and a score. In the illustrated embodiment of FIG. 15B, the line of weakness 1108A includes perforations 1110B.

In some embodiments, the plurality of absorbent articles 1501, 1601 of the absorbent system 1520 form a reel (not shown). The line of weakness 1108B may help to detach the plurality of absorbent articles 1501, 1601 from each other. Specifically, the plurality of absorbent articles 1501, 1601 may be torn along the line of weakness 1108B.

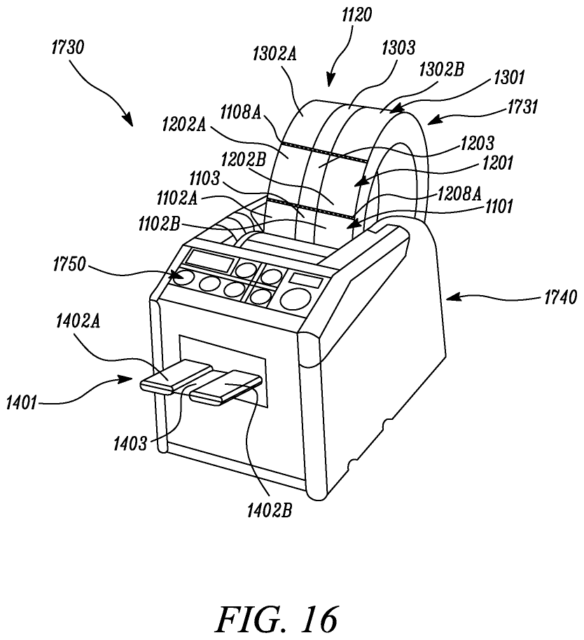

FIG. 16 illustrates a dispensing system 1730. The dispensing system 1730 includes the absorbent system 1120. The plurality of absorbent articles 1101, 1201, 1301, 1401 of the absorbent system 1120 forms a reel 1731. In some other embodiments, the dispensing system 1730 may include the absorbent system 1520 (shown in FIG. 15B).

The dispensing system 1730 further includes a dispenser 1740. In some embodiments, the dispenser 1740 at least partially receives the absorbent system 1120 therein, such that at least one absorbent article 1401 of the plurality of absorbent articles 1101, 1201, 1301, 1401 at least partially extends outwardly from the dispenser 1740. The absorbent article 1401 is substantially similar to the plurality of absorbent articles 1101, 1201, 1301 and includes at least two absorbent pads 1402A, 1402B spaced apart from each other and an interconnect member 1403.

In some embodiments, the dispenser 1740 may be a manual dispenser. As shown in FIG. 16, the dispenser 1740 is an automatic dispenser. The automatic dispenser may dispense a number of absorbent articles, as required, based on an input provided by the dental clinician. In some embodiments, the input may be received at one or more buttons 1750. In some embodiments, the one or more buttons 1750 may be disposed on the dispenser 1740. In some embodiments, the one or more buttons 1750 may not be disposed on the dispenser 1740.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. An absorbent article comprising:
at least two absorbent pads spaced apart from each other, each absorbent pad including an absorbent material configured to absorb a liquid, each absorbent pad further including an outer surface; and
an interconnect member joining the outer surfaces of the at least two absorbent pads, wherein the interconnect member is stretchable.

2-6. (canceled)

7. The absorbent article of claim 1, wherein each absorbent pad further comprises:
a first layer made of a liquid permeable material;
a second layer; and
a third layer disposed between the first layer and the second layer, the third layer comprising the absorbent material,
wherein the first layer and the second layer together define the outer surface, and wherein the third layer is enclosed by the first layer and the second layer.

8. The absorbent article of claim 7, wherein each absorbent pad further comprises a seal formed between the first layer and the second layer.

9. The absorbent article of claim 7, wherein the first layer is hydrophilic.

10. The absorbent article of claim 7, wherein the second layer is made of a liquid permeable material.

11. The absorbent article of claim 7, wherein the second layer is made of a liquid impermeable material.

12-22. (canceled)

23. The absorbent article of claim 1, wherein at least one of the absorbent pads further comprises a tube in fluid communication with the absorbent material, wherein the tube is configured to be attached to a suction source.

24. (canceled)

25. The absorbent article of claim 1, wherein each absorbent pad is arc-shaped, such that a first absorbent pad of the at least two absorbent pads forms an inner arc and a second absorbent pad of the at least two absorbent pads forms an outer arc.

26. The absorbent article of claim 25, further comprising an inner member attached to the first absorbent pad at least partially along the inner arc, wherein the inner member is made of a liquid impermeable material.

27-30. (canceled)

31. The absorbent article of claim 1, wherein each absorbent pad further comprises a film made of a liquid permeable material, wherein the film is folded upon itself to form three or more folded layers, and wherein the absorbent material is disposed within each absorbent pad.

32. The absorbent article of claim 31, wherein the film further forms a hollow core, and wherein the absorbent material is disposed within the hollow core.

33. The absorbent article of claim 31, wherein the absorbent material is disposed between at least two adjacent folded layers from the three or more folded layers.

34. The absorbent article of claim 31, wherein the film of each absorbent pad comprises a first tapered portion and a second tapered portion opposite to the first tapered portion, such that the film fully encloses the absorbent material, and wherein each absorbent pad further comprises:
at least a first seal configured to seal the film at the first tapered portion; and
at least a second seal configured to seal the film at the second tapered portion.

35-39. (canceled)

40. An absorbent system comprising:
a plurality of absorbent articles, each of the plurality of absorbent articles comprising:
at least two absorbent pads spaced apart from each other, each absorbent pad including an absorbent material configured to absorb a liquid, each absorbent pad further including an outer surface; and
an interconnect member joining the outer surfaces of the at least two absorbent pads, wherein the interconnect member is stretchable; and
at least one line of weakness, wherein the at least one line of weakness joins two adjacent absorbent articles of the plurality of absorbent articles.

41. (canceled)

42. The absorbent system of claim 40, wherein the at least one line of weakness joins the at least two absorbent pads and the interconnect member of one absorbent article to the at least two absorbent pads and the interconnect member of an adjacent absorbent article.

43-45. (canceled)

46. The absorbent system of claim 40, wherein the interconnect member is made of a liquid impermeable material.

47-49. (canceled)

50. The absorbent system of claim 40, wherein each absorbent pad further comprises:
a first layer made of a liquid permeable material;
a second layer; and
a third layer disposed between the first layer and the second layer, the third layer comprising the absorbent material,
wherein the first layer and the second layer together define the outer surface, and wherein the third layer is enclosed by the first layer and the second layer.

51-56. (canceled)

57. A dispensing system comprising:
an absorbent system, the absorbent system comprising:
a plurality of absorbent articles, each of the plurality of absorbent articles comprising:
at least two absorbent pads spaced apart from each other, each absorbent pad including an absorbent material configured to absorb a liquid, each absorbent pad further including an outer surface; and
an interconnect member joining the outer surfaces of the at least two absorbent pads, wherein the interconnect member is stretchable; and
at least one line of weakness, wherein the at least one line of weakness joins two adjacent absorbent articles of the plurality of absorbent articles; and
a dispenser, wherein the dispenser at least partially receives the absorbent system therein, such that at least one absorbent article of the plurality of absorbent articles at least partially extends outwardly from the dispenser.

58-64. (canceled)

65. The dispensing system of claim 57, wherein each absorbent pad further comprises:
a first layer made of a liquid permeable material;
a second layer; and
a third layer disposed between the first layer and the second layer, the third layer comprising the absorbent material,
wherein the first layer and the second layer together define the outer surface, and wherein the third layer is enclosed by the first layer and the second layer.

66-71. (canceled)
